(19) United States
(12) Patent Application Publication
(10) Pub. No.: US 2013/0177522 A1
(43) Pub. Date: Jul. 11, 2013

(54) NON-IRRITATING OPTHALMIC POVIDONE-IODINE COMPOSITIONS

(71) Applicants: Bo Liang, East Brunswick, NJ (US); Joseph A. Capriotti, Christiansted, VI (US); C. Michael Samson, New York, NY (US); Jason Stein, New York, NY (US); Michael Weiser, New York, NY (US)

(72) Inventors: Bo Liang, East Brunswick, NJ (US); Joseph A. Capriotti, Christiansted, VI (US); C. Michael Samson, New York, NY (US); Jason Stein, New York, NY (US); Michael Weiser, New York, NY (US)

(73) Assignee: FORESIGHT BIOTHERAPEUTICS, INC., New York, NY (US)

(21) Appl. No.: 13/515,987

(22) PCT Filed: Dec. 15, 2010
(86) PCT No.: PCT/US10/60489

§ 371 Date: Mar. 21, 2013

Related U.S. Application Data
(60) Provisional application No. 61/286,697, filed on Dec. 15, 2009.

(51) Int. Cl. A61K 47/10; A61K 47/08; A61K 9/00; A61K 31/79; A61K 45/06

(52) U.S. Cl. CPC A61K 47/10; A61K 31/79; A61K 45/06; A61K 9/0048; A61K 47/08
USPC 424/78.04

(57) ABSTRACT

Disclosed are compositions and methods comprising povidone-iodine and a cooling-effective amount of a chemical agent. The compositions are useful to relieve mild ocular irritation, enhance ocular comfort, and to provide a refreshing effect and improved sensation, when the povidone-iodine solution is applied to the eye.

### BACKGROUND

Ophthalmic compositions used for treatment of eye redness, ocular symptoms of allergies, and microbial infection are often irritating to the eye upon instillation. For example, certain iodine-containing ophthalmic compositions can be irritating to the eye upon instillation.

The use of a cooling agent, such as menthol, to provide a cooling effect on the skin and in the oral cavity is known. Cooling agents have also been added to food products such as chewing gum or mints, as well as to cigarettes, in order to provide a sensation of “coolness or freshness” during consumption. Menthol has also been added to topical pharmaceutical compositions to alleviate the sensation of inflammation and itch associated with bug bites and mild abrasions.

The sensation of coolness on the skin and mucosal surfaces resulting from the application of menthol is believed to be due to a specific action on sensory nerve endings. It is believed that cooling agents such as menthol exert their effect on cold receptors by interfering with the mobility of calcium ions across the cell membrane. Certain preparations of menthol, for example, have been perceived as being irritating to the eye, and consequently, menthol has not been utilized extensively in ophthalmic preparations.

### BRIEF SUMMARY OF THE INVENTION

Disclosed herein is an ophthalmic preparation comprising povidone-iodine at a concentration from about 0.1% to about 2.5%, a lubricant and/or a cooling agent. The lubricant and/or cooling agent are present in the preparation at a concentration which is not irritating to the eye. Optionally, an ophthalmic preparation also contains one or more of camphor, borneol, a lubricant, an emollient, a steroidal anti-inflammatory compound, and a non-steroidal anti-inflammatory compound.

In an aspect, PVP-I is present at a concentration of 0.2 to 2.0%, 0.3% to 1.5%, 0.36% to 1.0%, and 0.4% to 0.75%. In another aspect, PVP-I is present at a concentration of about 0.05%, about 0.1%, about 0.2%, about 0.3%, about 0.4%, about 0.5%, about 0.6%, about 0.7%, about 0.8%, about 0.9% and about 1.0%.

In an embodiment, the ophthalmic preparation includes a non-steroidal anti-inflammatory compound such as ketotifen fumarate, diclofenac sodium, nepafenac, bromfenac, flurbiprofen sodium, suprofen, celecoxib, naproxen, rofecoxib, and any combination thereof.

In another embodiment, the ophthalmic preparation includes a steroidal anti-inflammatory compound such as dexamethasone, dexamethasone alcohol, dexamethasone sodium phosphate, fluromethalone acetate, fluromethalone alcohol, lotoprendol etabonate, medrysone, prednisolone acetate, prednisolone sodium phosphate, difluprednate, rimexolone, hydrocortisone, hydrocortisone acetate, lodoxamide tromethamine, and any combination thereof.

In an aspect, the ophthalmic preparation comprises at least one viscosity increasing agent. A viscosity increasing agent may include polyvinyl alcohol, polyvinylpyrrolidone, methyl cellulose, hydroxypropylmethylcellulose, hydroxyethylcellulose, carboxymethylcellulose, hydroxypropylcellulose, and any combination thereof.

In an aspect, the ophthalmic preparation comprises at least one artificial tears-based lubricant. An artificial tears-based lubricant may include propylene glycol, glycerin, polyethylene glycol, dextran, blended polyvinyl alcohols, polyvinyl alcohol, polyethylene glycol, light mineral oil, hydroxypropyl methylcellulose, hypromellose, carbopol, carbomer 940 (polyacrylic acid), polyvinyl pyrrolidone, white petrolatum, soy lecithin, and sodium carboxyl methylcellulose.

In an aspect, the ophthalmic preparation comprises at least one bioadhesive agent. A bioadhesive agent may include polyvinylpyrrolidone (PVP), xanthan gum, locust bean gum, acacia gum, hydroxypropyl methylcellulose (HPMC), sodium alginate, pectin, gelatin, carbomer, polyvinylalcohol, gellan gum, tragacanth, acacia, and sodium carboxymethyl cellulose.

Disclosed herein is a method for treating and/or prophylaxis of an eye disorder or a microorganism infection of at least one tissue of the eye comprising administration of one or more doses of an ophthalmic preparation as disclosed herein to an eye.

In one aspect, a method includes prophylaxis of infection following corneal abrasion or ocular surgery.

In an aspect, a method is used to treat a disorder such as conjunctivitis, corneal abrasion, ulcerative infectious keratitis, epithelial keratitis, stromal keratitis, herpesvirus-related keratitis, ocular surface irregularity, tear deficiency, dry syndrome, meibomian gland disfunction, blepharitis, uveitis, and a microorganism infection of at least one tissue of the eye.

Disclosed herein is a method for treating and/or prophylaxis of a microorganism infection of a non-ophthalmic tissue, comprising contacting the tissue with a composition as disclosed herein.

### DETAILED DESCRIPTION OF THE INVENTION

The invention provides, in part, ophthalmic compositions comprising povidone-iodine in the range of about 0.01% to about 10% (weight/weight or weight/volume) and a cooling effective amount of a chemical agent to relieve mild ocular irritation, enhance ocular comfort, and to provide a refreshing effect and improved sensation, when the povidone-iodine solution is applied to the eye. Such an agent includes different chemical classes, including, but not limited to, cooling agents such as menthol, menthol derivatives including methone glycerin acetyl and menthyl esters, carboxamides, menthane glycerol ketals, alkyl substituted ureas, sulfonamides, terpene analogs, furanones, and phosphine oxides; or camphor, and borneol.

As will be understood by the skilled artisan, various cooling agents may have different properties, and the amount and type of cooling agent to use may depend upon the components of a desired composition, as well as the desired therapeutic or soothing effect, or the degree of the effect, sought. Cooling agents may be used in a concentration range from about 0.001% to about 10%, about 0.005% to about 10%, about 0.01% to about 10%, about 0.0.5% to about 10%, about 0.1% to about 10%, about 0.25% to about 9%, about 0.5% to about 8%, about 0.75% to about 7%, about 0.9% to about 6%, or about 1.0% to about 5.0%. In an embodiment, a cooling agent is present in a composition at a level of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%. In an embodiment, a cooling agent is present in a composition at a level of 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 3%, 4%, or 5%.

The ophthalmic composition may further comprise an artificial tear-based lubricant to improve the comfort. Artificial-tear based lubricants include, but are not limited to, propylene glycol, glycerin, polyethylene glycol, dextran, blended polyvinyl alcohols, polyvinyl alcohol, polyethylene glycol, light mineral oil, hydroxypropyl methylcellulose, hypromellose, carbopol, carbomer 940 (polyacrylic acid), polyvinyl pyrrolidone, white petrolatum, soy lecithin, and sodium carboxyl methylcellulose, as well as other agents known to those skilled in the art, or any combination thereof. Typically, such lubricants are employed at a level of from 0.1% to 2% by weight. In an embodiment, the lubricants are 1.0% propylene glycol, 0.3% glycerin, 2.7% blended polyvinyl alcohols, 1% polyvinyl alcohol, 1% polyethylene glycol, light mineral oil, 0.3% hydroxypropyl methylcellulose, 1.0% soy lecithin, 0.25% or 0.5% sodium carboxyl methylcellulose. In an embodiment, a lubricant is present in a composition at a level of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%. In an embodiment, a lubricant is present in a composition at a level of about 0.1%, 0.2%, 0.3%, 0.4%, 0.5%, 0.6%, 0.7%, 0.8%, 0.9%, about 1.0%, about 1.1%, about 1.2%, 1.3%, 1.4%, 1.5%, 1.6%, 1.7%, 1.8%, 1.9%, or 2.0%.

In an embodiment, a composition comprises povidone-iodine (PVP-I) at a concentration in the range of about 0.1% to about 2.5%. In another embodiment, a composition comprises povidone-iodine (PVP-I) at a concentration in the range between 0.2 and 1.5%, and in yet another embodiment, between 0.3% and 1.0%. In an embodiment, a composition comprises PVP-I at a concentration in the range of about 0.2 to about 2.0%, about 0.3% to about 1.5%, about 0.36% to about 1.0%, and about 0.4% to about 0.75%. In an embodiment, a composition comprises PVP-I at a concentration of about 0.05%, about 0.1%, about 0.2%, about 0.3%, about 0.4%, about 0.5%, about 0.6%, about 0.7%, about 0.8%, about 0.9% or about 1.0%. In an embodiment, a composition comprises povidone-iodine PVP-I at a concentration of 0.05%, 0.1%, 0.2%, 0.3%, 0.4%, 0.5%, 0.6%, 0.7%, 0.8%, 0.9% or 1.0%. In another embodiment, a composition comprises PVP-I at a concentration of about 2%, about 3%, about 4%, about 5%, about 6%, about 7%, about 8%, about 9% or about 10%.

In another embodiment of the invention, a composition is provided, comprising. povidone-iodine at a concentration from about 0.1% to about 10%, a lubricant, and a cooling agent at a concentration which is non-irritating to a non-ophthalmic tissue. Optionally, the composition may further comprise one or more of camphor, borneol, a lubricant, an emollient, a steroidal anti-inflammatory compound, and a non-steroidal anti-inflammatory compound. In yet another embodiment, an ophthalmic composition set forth herein is useful for a non-ophthalmic application.

### Methods

In an aspect, a composition of the invention is useful in the treatment of infections of the conjunctiva and cornea. In another aspect, the broad spectrum antimicrobial activity of povidone-iodine enables a composition of the invention to be used to treat ocular conjunctival or corneal infection caused by mycobacteria, viruses, fungi, and amoeba. Additionally the composition is useful in the infectious prophylaxis of patients recovering from ophthalmic surgery. There are no currently available povidone-iodine solutions that are comfortable for repeat application in the eye. The present invention provides, in part, compositions that meet this need.

In an embodiment of the invention, an ophthalmic composition is provided that is suitable for topical administration to an eye, effective for treatment and/or prophylaxis of a microorganism infection or a disorder of at least one tissue of the eye. Prophylaxis may be, for example, prophylaxis from infection following surgery, prophylaxis from infection after birth for the newborn, or prophylaxis from accidental contact with contaminating material. Accidental contact with contaminating material may occur, for example, during surgery or during food processing.

Surprisingly, it was discovered that the compositions set forth herein, comprising povidone-iodine combined with cooling agents set forth herein, and/or camphor, and/or borneol, and/or lubricants, and/or emollients, when present in a suitable pH range, eliminated the undesired irritating effect of PVP-I to the eye.

In an embodiment, an ophthalmic composition may further comprise one or more of (1) a penetration enhancer which enhances the penetration of povidone-iodine into the tissues of the eye (this may be a topical anesthetic) (2) a co-solvent or a nonionic surface agent—surfactant, which, for example, may be about 0.01% to 2% by weight; (3) a viscosity increasing agent, which, for example, may be about 0.01% to 2% by weight; and (4) a suitable ophthalmic vehicle.

The ophthalmic composition may be in the form of a solution, a suspension, an emulsion, a preparation, an ointment, a cream, a gel, or a controlled-release/sustain-release vehicle. By way of a non-limiting example, the composition may be in the form of a contact lens solution, eyewash, eyedrop, and the like.

In an aspect, the ophthalmic composition may be used for treatment and/or prophylaxis of a microorganism infection. The microorganism may be a bacterium, a virus, a fungus, or an amoeba, a parasite, or a combination thereof. In an embodiment, the bacteria may be a mycobacterium.

In an aspect, an ophthalmic composition may be used to treat a disorder such as, but not limited to, conjunctivitis, corneal abrasion, ulcerative infectious keratitis, epithelial keratitis, stromal keratitis, herpesvirus-related keratitis, ocular surface irregularity, tear deficiency, dry syndrome, meibomian gland dysfunction, blepharitis and uveitis. In another aspect, an ophthalmic composition may be used for prophylaxis of disorders such as conjunctivitis, corneal abrasion, ulcerative infectious keratitis, epithelial keratitis, stromal keratitis, herpesvirus-related keratitis, ocular surface irregularity, tear deficiency, dry syndrome, meibomian gland dysfunction, blepharitis and uveitis.

In another embodiment, the invention is directed to a method for treating and/or prophylaxis of an eye disorder or a microorganism infection of at least one tissue of the eye comprising the step of administering one of more doses of an ophthalmic composition, discussed above, to the eye. The eye disorder may be, for example, a microorganism infection of at least one tissue of the eye, conjunctivitis, corneal abrasion, ulcerative infectious keratitis, epithelial keratitis, stromal keratitis, herpes virus-related keratitis, ocular surface irregularity, tear deficiency, dry syndrome, meibomian gland dysfunction, and blepharitis. The microorganism may be bacteria (e.g., mycobacteria), virus, fungi, or amoebae.

In an embodiment, the dose volume administered to a subject may be between about 10 microliters and about 200 microliters, in another embodiment, between about 20 microliters and 100 microliters, and in another embodiment, between about 50 microliters and about 80 microliters, or about one drop per eye. Two or more drops may be added to an eye. Treatment or soothing of an eye may be effected by adding a single drop of composition disclosed herein, or by adding two or more drops, as required to achieve the desired result.

In an embodiment, administration frequency may be between 1 and 24 times a day. In an embodiment, administration frequency may be between 1 and 48 times a day. In another embodiment, administration frequency may be between 2 and 24 times a day. In another embodiment, administration frequency may be between 2 and 4 times a day. In another embodiment, administration frequency may be twice a day. In another embodiment, administration frequency may be once a day. In another embodiment, administration frequency may be less frequent than once a day. In another embodiment, administration frequency may be on demand, as therapeutic or soothing treatment is required or desired.

In an embodiment, a composition disclosed herein is used for prophylaxis and/or treatment of a non-ophthalmic tissue by contacting the tissue with the composition.

### Additional Compositions

Compositions and preparations disclosed herein may further comprise one or more non-steroidal anti-inflammatory compounds. Non-steroidal anti-inflammatory compounds include, but are not limited to, ketotifen fumarate, diclofenac sodium, nepafenac, bromfenac, flurbiprofen sodium, suprofen, celecoxib, naproxen, rofecoxib, and any combination thereof. Compositions and preparations disclosed herein may further comprise one or more steroidal anti-inflammatory compounds. Steroidal anti-inflammatory compounds include, but are not limited to dexamethasone, dexamethasone alcohol, dexamethasone sodium phosphate, fluromethalone acetate, fluromethalone alcohol, lotoprendol etabonate, medrysone, prednisolone acetate, prednisolone sodium phosphate, difluprednate, rimexolone, hydrocortisone, hydrocortisone acetate, lodoxamide tromethamine, and any combination thereof. Steroidal and non-steroidal compounds may be combined in a single composition or preparation contemplated or disclosed herein. In an embodiment, a steroidal anti-inflammatory compound or a non-steroidal anti-inflammatory compound is present in the composition or preparation at a level of about 0.01% to about 10%. In an embodiment, a steroidal anti-inflammatory compound or a non-steroidal anti-inflammatory compound is present in the composition or preparation at a level of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

The compositions and preparations disclosed herein can be administered as solutions, suspensions, emulsions (dispersions), gels, creams, or ointments in a suitable ophthalmic vehicle. In any of the compositions of this disclosure for topical administration, such as topical administration to the eye, the mixtures are preferably formulated as aqueous solutions at a pH of 3.5 to 6.5. Preferentially the pH was adjusted to between 4 and 5. This pH range may be achieved by the addition of acids/bases to the solution.

In an embodiment, an ophthalmic composition may comprise an optional co-solvent. In another embodiment, the solubility of the components of the present compositions may be enhanced by a surfactant or other appropriate co-solvent in the composition. Such co-solvents or surfactants include polysorbate-20, -60, and -80, a polyoxyethylene/polyoxypropylene surfactant (e.g. Pluronic F-68, F-84 and P-103), cyclodextrin, tyloxapol, PEG 35 Castor oil (Cremophor EL), polyoxyl 40 Stearate (Myrj 52), other agents known to those skilled in the art, or a combination thereof. Typically, such co-solvents are present at a level of from about 0.01% to about 2% by weight.

In an embodiment, a composition may comprise an optional agent that can increase viscosity. As will be understood by the skilled artisan when armed with the present disclosure, it may be desirable to increase viscosity above that of a simple aqueous solution in order to increase ocular absorption of the active compound, to decrease variability in dispensing the formulation, to decrease physical separation of components of a suspension or emulsion of the formulation and/or to otherwise improve the ophthalmic formulation. Such viscosity-enhancing agents include, but are not limited to, polyvinyl alcohol, polyvinyl pyrrolidone, methyl cellulose, hydroxypropylmethyl cellulose, hydroxyethyl cellulose, carboxymethyl cellulose, hydroxypropyl cellulose, other agents known to those skilled in the art, or any combination thereof. Such agents are typically employed at a level of from about 0.01% to about 2% by weight.

In another aspect, bioadhesive agents may comprise the compositions, in order to increase the retention time of the drug gradient over a biological substrate. The bioadhesive agents include, but are not limited to, polyvinylpyrrolidone (PVP), xanthan gum, locust bean gum, acacia gum, hydroxypropyl methylcellulose (HPMC), sodium alginate, pectin, gelatin, carbomer, polyvinylalcohol, gellan gum, tragacanth, acacia, and sodium carboxymethyl cellulose, as well as other agents known to those skilled in the art, or any combination thereof. In yet another embodiment, compositions of the invention may comprise viscoelastic agents such as methyl cellulose, carboxymethyl cellulose, hydroxyethyl cellulose, polyvinyl alcohol, dextran, chondroitin sulfate and salts thereof, and hyaluronic acid and salts thereof.

In another aspect, compositions of the invention may comprise one or more buffering agents, isotonizing agents, solubilizers, stabilizers, chelating agents, and any combinations thereof. Such additional components may be used at concentrations that provide enhanced comfort or therapeutic properties to the PVP-I compositions disclosed herein. In another aspect, such additional components may be used at concentrations in which the additional component itself has a therapeutic and/or soothing effect, in addition to the effect obtained from the PVP-I compositions disclosed herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### Preparation of Non-Irritating PVP-I Ophthalmic Solution 1

By way of a non-limiting example, a PVP-I ophthalmic solution is prepared using 0.36%, 0.48%, or 0.6% PVP-I, by weight, as desired in the final product, and combining with 1.8% povidone, ethanol (0.1%), boric acid, camphor, poloxamer 407, polysorbate 80, potassium chloride, sodium borate, sodium chloride, and purified water.

### Example 2

### Preparation of Non-Irritating PVP-I Ophthalmic Solution 2

By way of a non-limiting example, a PVP-I ophthalmic solution is prepared using 0.36%, 0.48%, or 0.6% PVP-I, by weight, as desired in the final product, and combining with 0.2% polysorbate 80, ethanol (0.1%), boric acid, edetate disodium, menthol, sodium borate, and purified water.

### Example 3

### Preparation of PVP-I Preserved Ophthalmic Solution 3

By way of a non-limiting example, a PVP-I ophthalmic solution is prepared using 0.36%, 0.48%, or 0.6% PVP-I, by weight, as desired in the final product, and combining with 0.5% carboxymethylcellulose sodium, boric acid, calcium chloride, magnesium chloride, sodium borate, sodium chloride, and purified water. In an embodiment, a preserved ophthalmic solution comprises hydrochloric acid and/or sodium hydroxide to adjust pH.

The invention has been described herein by reference to certain embodiments. However, as variations thereof will become apparent to those skilled in the art, when armed with the disclosure set forth herein, the invention is not to be considered as limited thereto. All patents, patent applications, and references cited herein are hereby incorporated by reference in their entirety.

1. A ophthalmic preparation comprising:
a. povidone-iodine at a concentration from about 0.1% to about 2.5% said ophthalmic preparation,
b. at least one member selected from the group consisting of a lubricant and a cooling agent, at a concentration which is not irritating to the eye; and
c. optionally, one or more of the members selected from the group consisting of camphor, borneol, a lubricant, an emollient, a steroidal anti-inflammatory compound, and a non-steroidal anti-inflammatory compound.

2. The ophthalmic preparation of claim 1, wherein the PVP-I is present at a concentration selected from the group consisting of 0.2% to 2.0%, 0.3% to 1.5%, 0.36% to 1.0%, and 0.4% to 0.75%.

3. The ophthalmic preparation of claim 1, wherein the PVP-I is present at a concentration selected from the group consisting of about 0.05%, about 0.1%, about 0.2%, about 0.3%, about 0.4%, about 0.5%, about 0.6%, about 0.7%, about 0.8%, about 0.9% and about 1.0%.

4. The ophthalmic preparation of claim 1, wherein said non-steroidal anti-inflammatory compound is selected from the group consisting of ketotifen fumarate, diclofenac sodium, nepafenac, bromfenac, flurbiprofen sodium, suprofen, celecoxib, naproxen, rofecoxib, and any combination thereof.

5. The ophthalmic preparation of claim 1, wherein said steroidal anti-inflammatory compound is selected from the group consisting of dexamethasone, dexamethasone alcohol, dexamethasone sodium phosphate, fluromethalone acetate, fluromethalone alcohol, lotoprendol etabonate, medrysone, prednisolone acetate, prednisolone sodium phosphate, difluprednate, rimexolone, hydrocortisone, hydrocortisone acetate, lodoxamide tromethamine, and any combination thereof.

6. The ophthalmic preparation of claim 1 wherein said preparation further comprises a viscosity increasing agent.

7. The ophthalmic preparation of claim 6 wherein said viscosity increasing agent is selected from the group consisting of polyvinyl alcohol, polyvinylpyrrolidone, methyl cellulose, hydroxypropylmethylcellulose, hydroxyethylcellulose, carboxymethylcellulose, hydroxypropylcellulose, and any combination thereof.

8. The ophthalmic preparation of claim 1, wherein the preparation comprises at least one artificial tears-based lubricant.

9. The ophthalmic preparation of claim 8, wherein said artificial tears-based lubricant is selected from the group consisting of propylene glycol, glycerin, polyethylene glycol, dextran, blended polyvinyl alcohols, polyvinyl alcohol, polyethylene glycol, light mineral oil, hydroxypropyl methylcellulose, hypromellose, carbopol, carbomer 940 (polyacrylic acid), polyvinyl pyrrolidone, white petrolatum, soy lecithin, sodium carboxyl methylcellulose, and any combination thereof.

10. The ophthalmic preparation of claim 1, further comprising at least one bioadhesive agent.

11. The ophthalmic preparation of claim 10, wherein said bioadhesive agent is selected from the group consisting of polyvinylpyrrolidone (PVP), xanthan gum, locust bean gum, acacia gum, hydroxypropyl methylcellulose (HPMC), sodium alginate, pectin, gelatin, carbomer, polyvinylalcohol, gellan gum, tragacanth, acacia, sodium carboxymethyl cellulose, and any combination thereof

12. A method for treating and/or prophylaxis of an eye disorder or a microorganism infection of at least one tissue of the eye comprising the step of administering one or more doses of the ophthalmic preparation of claim 1 to said eye.

13. The method of claim 12 wherein said prophylaxis is prophylaxis of infection following corneal abrasion or ocular surgery.

14. The method of claim 12 wherein said eye disorder is selected from the group consisting of conjunctivitis, corneal abrasion, ulcerative infectious keratitis, epithelial keratitis, stromal keratitis, herpesvirus-related keratitis, ocular surface irregularity, tear deficiency, dry syndrome, meibomian gland disfunction, blepharitis, uveitis, and a microorganism infection of at least one tissue of the eye.

15. A method for treating and/or prophylaxis of a microorganism infection of a tissue comprising the step of contacting a desired tissue with one or more doses of the ophthalmic preparation of claim 1.
